(19) United States
(12) Plant Patent Application Publication
(10) Pub. No.: US 2024/0349634 P1
(43) Pub. Date: Oct. 17, 2024

(54) LOBELIA PLANT NAMED 'WNLOBLCBE23'

(50) ;

(71) Applicant: BENJAMIN K. WINSLOW, Austin, TX (US)

(72) Inventor: BENJAMIN K. WINSLOW, Austin, TX (US)

(73) Assignee: WINGEN, LLC, Mustang Ridge, TX (US)

(21) Appl. No.: 18/135,166

(22) Filed: Apr. 16, 2023

(51) Int. Cl. A01H 6/26

(52) U.S. Cl. CPC A01H 6/268
USPC PLT/451

(57) ABSTRACT

A new and distinct cultivar of Lobelia plant named ‘WNLOBLCBE23’, characterized by its compact, upright to outwardly spreading and mounding to trailing plant habit; vigorous and sturdy growth habit; freely branching habit; dense and bushy plant form; freely flowering habit; long flowering period; large purplish blue-colored flowers with white-colored centers; long flowering period; relative high temperature tolerance; and good container and garden performance.

Botanical designation: Lobelia erinus.

Cultivar denomination: ‘WNLOBLCBE23’.

### BACKGROUND OF THE INVENTION

The present invention relates to a new and distinct cultivar of Lobelia plant, botanically known as Lobelia erinus and hereinafter referred to by the name ‘WNLOBLCBE23’.

The new Lobelia plant is a product of a planned breeding program conducted by the Inventor in Alajuela, Costa Rica and Twinsburg, Ohio. The objective of the breeding program is to create new outwardly spreading, freely-branching and bushy Lobelia plants with a long flowering period, high temperature tolerance and good garden performance.

The new Lobelia plant originated from a cross-pollination made by the Inventor in Alajuela, Costa Rica on Nov. 16, 2016 of Lobelia erinus ‘Wesloba’, disclosed in U.S. Plant Pat. No. 15,872, as the female, or seed, parent with Lobelia erinus ‘Bella Mare’, not patented, as the male, or pollen, parent. The new Lobelia plant was discovered and selected by the Inventor as a single flowering plant within the progeny of the stated cross-pollination in a controlled greenhouse environment in Twinsburg, Ohio on Aug. 19, 2017.

Asexual reproduction of the Lobelia plant by vegetative terminal cuttings in Twinsburg, Ohio since Aug. 25, 2017 has shown that the unique features of this new Lobelia plant are stable and reproduced true to type in successive generations.

### SUMMARY OF THE INVENTION

Plants of the new Lobelia have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘WNLOBLCBE23’. These characteristics in combination distinguish ‘WNLOBLCBE23’ as a new and distinct Lobelia plant:
1. Compact, upright to outwardly spreading and mounding to trailing plant habit.
2. Vigorous and sturdy growth habit.
3. Freely branching habit; dense and bushy plant form.
4. Freely flowering habit.
5. Long flowering period.
6. Large purplish blue-colored flowers with white-colored centers.
7. Long flowering period.
8. Relatively high temperature tolerant.
9. Good container and garden performance.

Plants of the new Lobelia differ primarily from plants of the female parent, ‘Wesloba’, in the following characteristics:
1. Plants of the new Lobelia are slightly more vigorous than plants of ‘Wesloba’.
2. Plants of the new Lobelia have larger flowers than plants of ‘Wesloba’.
3. Flowers of plants of the new Lobelia are lighter purplish blue in color than flowers of plants of ‘Wesloba’.
4. White-colored flower centers of plants of the new Lobelia are larger than white-colored flower centers of plants of ‘Wesloba’.

Plants of the new Lobelia differ primarily from plants of the male parent, ‘Bella Mare’, in the following characteristics:
1. Plants of the new Lobelia are more vigorous than and not as compact as plants of ‘Bella Mare’.
2. Plants of the new Lobelia have larger flowers than plants of ‘Bella Mare’.
3. Flowers of plants of the new Lobelia are darker purplish blue in color than flowers of plants of ‘Bella Mare’.
4. Plants of the new Lobelia are more high temperature tolerant than plants of ‘Bella Mare’.

Plants of the new Lobelia can be compared to plants of Lobelia x hybrida ‘Lobetis’, disclosed in U.S. Plant Pat. No. 16,112. In side-by-side comparisons, plants of the new Lobelia differ primarily from plants of ‘Lobetis’ in the following characteristics:
1. Plants of the new Lobelia are stronger and sturdier than plants of ‘Lobetis’.
2. Plants of the new Lobelia have larger flowers than plants of ‘Lobetis’.
3. Flowers of plants of the new Lobelia are slightly darker purplish blue in color than flowers of plants of ‘Lobetis’.
4. Plants of the new Lobelia are more high temperature tolerant than plants of ‘Lobetis’.

### BRIEF DESCRIPTION OF THE PHOTOGRAPHS

The accompanying colored photographs illustrate the overall appearance of the new Lobelia plant showing the colors as true as it is reasonably possible to obtain in colored reproductions of this type. Colors in the photographs may differ slightly from the color values cited in the detailed botanical description which accurately describe the actual colors of the new Lobelia plant.

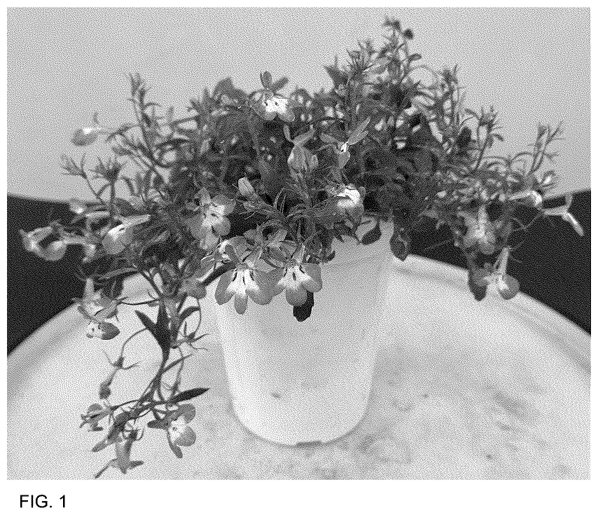

The photograph on the first sheet (FIG. 1) is a side perspective view of a typical flowering plant of ‘WNLOBLCBE23’ grown in a container.

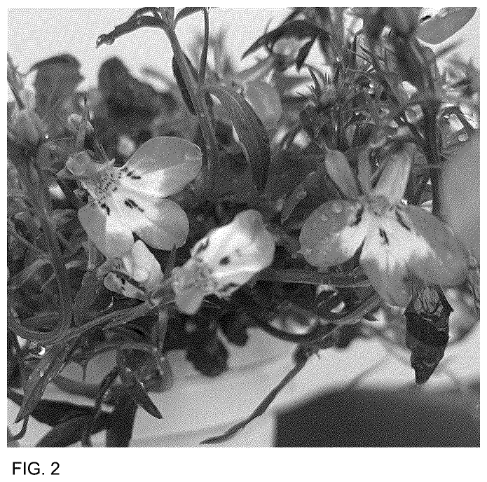

The photograph on the second sheet (FIG. 2) is a close-up view of a typical flowering plant of ‘WNLOBLCBE23’.

### DETAILED BOTANICAL DESCRIPTION

The aforementioned photographs and following observations and measurements describe plants grown during the summer in 740-ml containers in an acrylic-covered greenhouse in Carlton, Michigan and under cultural practices typical of commercial Lobelia production. During the production of the plants, day temperatures ranged from 18C. to 32C. and night temperatures ranged from 18C. to 24C. Plants were ten weeks from planting rooted cuttings when the photographs and description were taken. In the following description, color references are made to The Royal Horticultural Society Colour Chart, 2015 Edition, except where general terms of ordinary dictionary significance are used.
Botanical classification: Lobelia erinus ‘WNLOBLCBE23’.
Parentage:
   Female, or seed, parent.—Lobelia erinus ‘Wesloba’, disclosed in U.S. Plant Pat. No. 15,872.
   Male, or pollen, parent.—Lobelia erinus ‘Bella Mare’, not patented.
Propagation:
   Type cutting.—Vegetative terminal cuttings.
   Time to initiate roots, summer.—About seven to twelve days at ambient temperatures about 28C.
   Time to initiate roots, winter.—About 10 to 14 days at ambient temperatures about 20C.
   Time to produce a rooted young plant, summer.—About three to four weeks at ambient temperatures about 28C.
   Time to produce a rooted young plant, winter.—About four to five weeks at ambient temperatures about 20C.
   Root description.—Fine, fibrous; typically white in color, actual color of the roots is dependent on substrate composition, water quality, fertilizer type and formulation, substrate temperature and physiological age of roots.
   Rooting habit.—Freely branching; medium density.
Plant description:
   Plant and growth habit.—Compact, upright to outwardly spreading and mounding to trailing plant habit; freely branching habit with lateral branches developing at potentially every node; dense and bushy plant habit; vigorous and sturdy growth habit and rapid growth rate.
   Plant height.—About 9 cm.
   Plant width.—About 24 cm.
   Lateral branch description.—Length: About 12 cm. Diameter: About 1 mm. Internode length: About 1.5 cm. Strength: Strong, flexible. Aspect: Upright to outwardly spreading to trailing. Texture and luster: Smooth, glabrous; somewhat glossy. Color: Close to 144A and 146A.
Leaf description:
   Arrangement.—Alternate, simple.
   Length.—About 2.6 cm.
   Width.—About 1.1 cm.
   Shape.—Obovate; slightly recurved.
   Apex.—Bluntly acute.
   Base.—Cuneate.
   Margin.—Crenate; not undulate.
   Texture and luster, upper and lower surfaces.—Smooth, glabrous; somewhat glossy.
   Venation pattern.—Pinnate.
   Color.—Developing leaves, upper surface: More green than 146A. Developing leaves, lower surface: Close to 146A. Fully expanded leaves, upper surface: Close to between 146A and 147A; venation, close to between 146A and 147A. Fully expanded leaves, lower surface: Close to 146A to 146B; venation, close to 146A to 146B.
   Petioles.—Length: About 3 mm. Diameter: About 1 mm. Texture and luster: Smooth, glabrous; somewhat glossy. Color, upper surface: Close to between 146A and 147A. Color, lower surface: Close to 146A to 146B.
Flower description:
   Flower arrangement, habit and shape.—Single, flowers axillary or terminal; flowers face mostly outwardly; freely flowering habit with flowers potentially developing at every axil; flowers bilabiate with two upper petals and three larger fused lower petals.
   Fragrance.—None detected.
   Natural flowering season.—Long flowering period, in Michigan, plants of the new Lobelia flower continuously from planting in the spring until frost in the autumn; early flowering habit, plants begin flowering about seven weeks after planting.
   Flower longevity on the plant.—Longevity of individual flowers is highly dependent on temperature, flowers typically last about one to two weeks on the plant; flowers persistent.
   Flower diameter.—About 1.75 cm by 2.1 cm.
   Flower depth.—About 1 cm.
   Flower throat diameter.—About 4 mm.
   Flower tube length.—About 9 mm to 10 mm.
   Flower tube diameter, distally.—About 4 mm.
   Flower tube diameter, proximally.—About 2 mm.
   Flower buds.—Length: About 7.5 mm. Diameter: About 3.5 mm. Shape: Oblong. Color, developing sepals: Close to 144B.
   Petals.—Arrangement: Single whorl of five petals fused towards the base; two upper petals and three larger fused lower petals. Upper petals: Lobe length, beyond throat: About 7 mm. Lobe width, beyond throat: About 2 mm. Shape: Oblanceolate. Apex: Cuspidate to mucronate. Margin: Entire; not undulate. Texture and luster, upper and lower surfaces: Smooth, glabrous; matte. Color, when opening and fully opened, upper surface: Close to 96B to 96C; towards the throat, close to NN155D; venation, close to 96B to 96C or NN155D; color becoming closer to 96C to 96D with subsequent development. Color, when opening and fully opened, lower surface: Close to 97A; venation, close to 97A or NN155D; color becoming closer to 97B with subsequent development. Lower petals: Lobe length, beyond throat: About 1 cm. Lobe width, beyond throat: About 6 mm. Shape: Obovate. Apex: Cuspidate to mucronate. Margin: Entire; not undulate. Texture and luster, upper and lower surfaces: Smooth, glabrous; matte. Color, when opening and fully opened, upper surface: Close to 96B to 96C; towards the throat, close to NN155D with nectar guides, close to 96A; venation, close to 96B to 96C or NN155D; color becoming closer to 96C to 96D with subsequent development. Color, when opening and fully opened, lower surface: Close to 97A; towards the tube, close to NN155D, nectar guides are visible from upper surface; venation, close to 97A or NN155D; color becoming closer to 97B with subsequent development. Color, throat: Close to 97A; spots, close to 93A to 93B. Color, tube: Close to 97A to 97B.
   Sepals.—Arrangement: Single whorl of five sepals, fused at the base; star-shaped calyx. Length: About 6 mm. Width: About 1 mm. Shape: Acicular. Apex: Acute. Margin: Entire; not undulate. Texture and luster, upper and lower surfaces: Smooth, glabrous; slightly glossy. Color, upper and lower surfaces: Close to 146A.
   Peduncles.—Length: About 1 cm. Diameter: Less than 1 mm. Strength: Strong, flexible; wiry. Aspect: About 45 to 90 degrees from lateral branch axis. Texture and luster: Smooth, glabrous; slightly glossy. Color: Close to 146A.
   Reproductive organs.—Stamens: Quantity per flower: Five. Filament length: About 5 mm. Filament color: Close to 97A. Anther size: About 0.5 mm by 2 mm. Anther shape: Oblong. Anther color: Close to 187A. Pollen amount: None observed. Pistils: Quantity per flower: One. Pistil length: About 8 mm. Stigma shape: Globose. Stigma color: Close to N187A. Style color: Close to 144A to 144B. Ovary color: Close to 144A.
   Fruits and seeds.—To date, fruit and seed development have not been observed on plants of the new Lobelia.
Pathogen & pest resistance: To date, plants of the new Lobelia have not been noted to be resistant to pathogens and pests common to Lobelia plants.
Garden performance: Plants of the new Lobelia have been observed to have good garden performance and to tolerate wind, rain and to be relatively tolerant to high temperature conditions.

It is claimed:

1. A new and distinct Lobelia plant named ‘WNLOBLCBE23’ as illustrated and described.
